(19) United States
(12) Patent Application Publication
(10) Pub. No.: US 2022/0240963 A1
(43) Pub. Date: Aug. 4, 2022

(54) SELF-INTERFACING JAW MEMBERS FOR SURGICAL INSTRUMENTS

(71) Applicant: Covidien LP, Mansfield, MA (US)

(72) Inventor: Brian A. Rockrohr, Guilford, CT (US)

(21) Appl. No.: 17/612,835

(22) PCT Filed: Jul. 14, 2020
(86) PCT No.: PCT/US2020/041872

§ 371 Date: Nov. 19, 2021

Related U.S. Application Data
(60) Provisional application No. 62/875,662, filed on Jul. 18, 2019.

(51) Int. Cl. A61B 17/29

(52) U.S. Cl. CPC A61B 17/29; A61B 2017/2926

(57) ABSTRACT

A surgical instrument end effector includes a first jaw member and a second jaw member. The first jaw member has a first tissue contacting surface. The first tissue contacting surface is configured to self-interface and includes a first portion and a second portion. The first portion includes a first plurality of teeth having a first tooth pitch. The second portion includes a second plurality of teeth having the first tooth pitch. The second plurality of teeth is offset from the first plurality of teeth by ½ of the first tooth pitch. The second jaw member includes a second tissue contacting surface. The second tissue contacting surface is positioned to enable the first and second tissue contacting surfaces to interface with one another.

### BACKGROUND

Many surgical procedures are performed through small incisions in an effort to reduce a patient's trauma and recovery time. Such procedures are generally referred to as minimally invasive. During a typical minimally invasive procedure, surgical instruments with end effectors, which may be robotic or handheld instruments, are inserted into the patient's body to access a target site in the patient's body. Some of these end effectors can include a pair of grasping jaw members.

### SUMMARY

According to one aspect, this disclosure is directed to a surgical instrument end effector. The end effector includes a first jaw member having a first tissue contacting surface. The first tissue contacting surface is configured to self-interface and includes a first portion and a second portion. The first portion includes a first plurality of teeth having a first tooth pitch. The second portion includes a second plurality of teeth having the first tooth pitch. The second plurality of teeth is offset from the first plurality of teeth by ½ of the first tooth pitch. The second jaw member includes a second tissue contacting surface. The second tissue contacting surface is positioned to enable the first and second tissue contacting surfaces to interface with one another.

In embodiments, the first and second jaw members may be movable relative to one another to cause the first and second tissue contacting surfaces to selectively interface with one another. The end effector may define a longitudinal axis, and the first and second portions of the first tissue contacting surface may be separated by a channel that extends longitudinally along the first jaw member.

In certain embodiments, the plurality of teeth of the first portion of the first tissue contacting surface may be spaced apart by a plurality of recesses defined in the first tissue contacting surface. The plurality of teeth of the second portion of the first tissue contacting surface may be spaced apart by a plurality of recesses defined in the first tissue contacting surface.

In embodiments, the first and second tissue contacting surfaces are substantially identical.

In some embodiments, the second tissue contacting surface includes first and second portions that may be separated by a channel extending longitudinally along the second jaw member.

In various embodiments, the first jaw member may define a slot that extends through the first tissue contacting surface of the first jaw member.

In embodiments, the first and second jaw members may be identical or substantially identical.

In some embodiments, the first and second jaw members may be configured to be manually controlled, robotically controlled, or combinations thereof

According to another aspect, this disclosure is directed to an end effector for a robotically controlled surgical instrument. The end effector includes a first jaw member having a first tissue contacting surface. The first tissue contacting surface is self-interfacing and includes a first portion and a second portion. The first portion includes a first plurality of teeth. The second portion includes a second plurality of teeth. The first and second plurality of teeth have the same tooth pitch. The first plurality of teeth is longitudinally separated from the second plurality of teeth by ½ pitch offset. The end effector includes a second jaw member that includes a second tissue contacting surface that is identical or substantially identical to the first tissue contacting surface of the first jaw member. The second tissue contacting surface is positioned to enable the first and second tissue contacting surfaces to interface with one another.

According to still another aspect, this disclosure is directed to a surgical instrument. The surgical instrument includes an end effector having a first jaw member and a second jaw member. Each of the first and second jaw members includes a tissue contacting surface that is configured to self-interface and is separated by a longitudinal channel. The tissue contacting surfaces of the first and second jaw members are positioned to interface with one another.

The details of one or more aspects of this disclosure are set forth in the accompanying drawings and the description below. Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of this disclosure and, together with a general description of this disclosure given above, and the detailed description of the embodiment(s) given below, serve to explain the principles of this disclosure, wherein:

FIG. 1 is a perspective view of an illustrative surgical instrument including an end effector in accordance with the present disclosure;

FIG. 1A is a side view of a distal portion of one end effector of the surgical instrument of FIG. 1, the end effector shown in a closed position, the end effector having two different jaw members with tissue grasping surfaces that interface with one another when the end effector is disposed in the closed position thereof;

FIG. 1B is a side view of a distal portion of another end effector of the surgical instrument of FIG. 1, the end effector shown in a closed position, the end effector having two of the same jaw members, the jaw members having tissue grasping surfaces that do not interface with one another when the end effector is disposed in the closed position thereof;

FIG. 2 is a perspective view of a distal portion of one embodiment of an end effector in accordance with the principles of this disclosure; and

FIG. 3 is an enlarged side view of a distal portion of the end effector of FIG. 2, the end effector having two of the same jaw members, each of the jaw members having the same self-interfacing tissue grasping surface.

### DETAILED DESCRIPTION

Embodiments of this disclosure are described in detail with reference to the drawings, in which like reference numerals designate identical or corresponding elements in each of the several views. As commonly known, the term “clinician” refers to a doctor (e.g., a surgeon), a nurse, or any other care provider and may include support personnel. Additionally, the term “proximal” refers to the portion of structure that is closer to the clinician and the term “distal” refers to the portion of structure that is farther from the clinician. As used herein, the term “same” refers to identical structure. Further, as used herein, the term “self-interfacing” or the like refers to structure that complements itself.

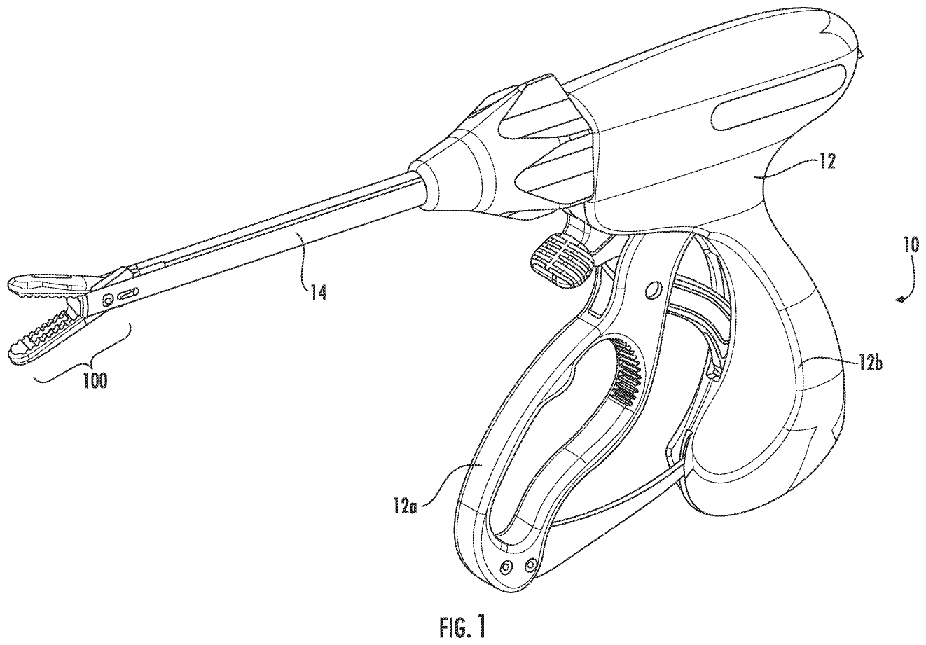

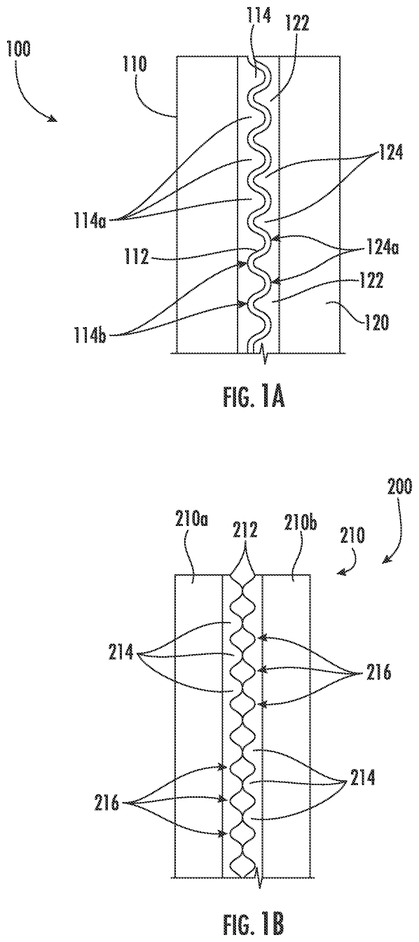

Embodiments of the end effectors and/or jaw members of this disclosure may be configured for use with any suitable handheld surgical instruments or systems, such as surgical instrument 10 illustrated in FIG. 1. Surgical instrument 10 generally includes a handle assembly 12, an elongate shaft assembly 14 that extends distally from handle assembly 12, and an end effector 100 supported on distal end portion of elongated shaft assembly 14. End effector 100 is operable upon an actuation of a movable handle 12a relative to a stationary handle 12b of handle assembly 12. For a detailed description of one exemplary handheld surgical instrument, or components thereof, reference may be made to U.S. Pat. No. 7,118,587, the entire contents of which are incorporated by reference herein.

The various embodiments disclosed herein may also be configured to work with robotic surgical systems and what is commonly referred to as “Telesurgery.” For a detailed description of one exemplary robotic surgical system, or components thereof, reference may be made to U.S. Pat. No. 10,179,413, the entire contents of which are incorporated by reference herein.

Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Pat. No. 8,828,023, and PCT Application Publication No. WO2016/025132, the entire contents of each of which are incorporated by reference herein.

In the following description, well-known functions or constructions are not described in detail to avoid obscuring this disclosure in unnecessary detail.

As seen in FIG. 1A, end effector 100 of surgical instrument 10, has two different jaw members, a first jaw member 110 and a second jaw member 120. Each jaw member has a different tissue contacting surface so that the tissue contacting surfaces of these different jaw members are positioned to interface with one another when end effector 100 is disposed in a closed position, but neither of first and second jaw members 110, 120 is self-interfacing.

The first jaw member 110 of end effector 100 includes a first tissue contacting surface 112 and the second jaw member 120 of end effector 100 has a second tissue contacting surface 122 that is configured to mate with first tissue contacting surface 112. In particular, first tissue contacting surface 112 of first jaw member 110 includes a plurality of teeth 114 that extends across a width of first jaw member 110. Each tooth 114a of the plurality of teeth 114 of first jaw member 110 is spaced apart by a recess 114b defined within first tissue contacting surface 112 between adjacent teeth of the plurality of teeth 114 of first jaw member 110. Similarly, second tissue contacting surface 122 of second jaw member 120 includes a plurality of teeth 124 that extends partially and/or completely across a width of second jaw member 120. Each tooth 124a of the plurality of teeth 124 of second jaw member 120 is spaced apart by a recess 124b defined within second tissue contacting surface 122 between adjacent teeth of the plurality of teeth 124 of second jaw member 120. Thus, when end effector 100 is disposed in a closed position as seen in FIG. 1A, recesses 124b of second tissue contacting surface 122 receive the plurality of teeth 114 of first jaw member 110 and recesses 114b of first tissue contacting surface 112 receive the plurality of teeth 124 of second jaw member 120 so that first and second jaw members 110, 120 interface or nest with one another.

As seen in FIG. 1B, another end effector that may be used with surgical instrument 10, generally referred to as end effector 200, includes two of the same jaw members. In particular, end effector 200 includes two identical jaw members 210 that are not self-interfacing. The two identical jaw members 210 include a first jaw member 210a disposed in an opposite direction to a second jaw member 210b so that the identical jaw members 210 face one another. Each of the first and second jaw members 210a, 210b includes a tissue contacting surface 212 with a plurality of teeth 214 spaced apart by a plurality of recesses 216. Thus, when end effector 200 is disposed in a closed position as seen in FIG. 1B, the plurality of teeth 214 of the first jaw member 210a aligns with the plurality of teeth of the second jaw member 210b, and the plurality of recesses 216 of the first jaw member 210a aligns with the plurality of recesses 216 of the second jaw member 210b. Accordingly, since first and second jaw members 210a, 210b cannot interface with one another, neither of first or second jaw members 210a, 210b is self-interfacing.

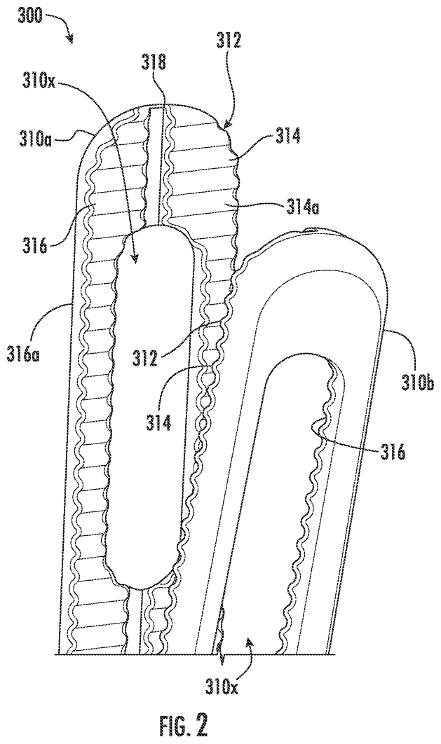

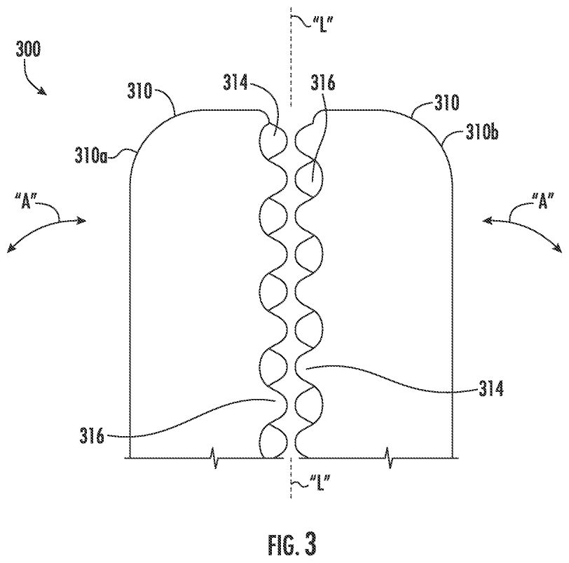

Turning now to FIGS. 2 and 3, one embodiment of a self-interfacing end effector for use with surgical instrument 10, in accordance with the present disclosure, is generally referred to as 300 and defines a longitudinal axis “L” that extends from a proximal end portion of end effector 300 toward a distal end portion of end effector 300. Self-interfacing end effector 300 includes two of the same jaw members 310 that are selectively movable (e.g., pivotable) relative to one another, as indicated by arrows “A,” to selectively interface with one another. In particular, end effector 300 includes a first jaw member 310a and a second jaw member 310b, each of which defines a central slot therethrough 310x. Each of the first and second jaw members 310a, 310b has a tissue contacting surface 312. The tissue contacting surfaces 312 of the first and second jaw members 310a, 310b have a common configuration that is self-interfacing such that these tissue contacting surfaces 312 will interdigitate when placed in an opposing orientation with one another. Each tissue contacting surface 312 includes a first portion 314 and second portion 316 separated by a channel 318 that extends longitudinally along tissue contacting surface 312. First portion 314 includes a first set of teeth 314a and second portion 316 includes a second set of teeth 316a. The first set of teeth 314a and the second set of teeth 316a have the same tooth pitch, but with the first set of teeth 314a longitudinally offset from the second set of teeth 316a by ½ pitch offset. The ½ pitch offset of the first and second sets of teeth 314a, 316a enable first portion 314 of first jaw member 310a to interface with second portion 316 of second jaw member 310b while second portion 316 of first jaw member 310a interfaces with first portion 314 of second jaw member 310b as the end effector 300 moves from an open position to a closed position.

In some embodiments, tissue contacting surface 312 may include any even number of portions (e.g., 4, 6, 8, etc.), each with the same and/or different tooth patterns, pitches, sizes, shapes, etc., or combinations thereof. For example, in one embodiment, tissue contacting surface 312 may include four portions, with each portion separated by a channel (e.g., three parallel channels extending longitudinally along tissue contacting surface) so that corresponding portions of the tissue contacting surfaces selectively interface with one another when two of the tissue contacting surfaces are arranged together to form an end effector (e.g., two of the same tissue contacting surfaces).

As can be appreciated, although the tissue contacting surfaces of disclosed jaw members may have a common configuration to allow them to interdigitate when placed in an opposing orientation (e.g., self-interfacing), other structures of the disclosed jaw members supporting those tissue contacting surfaces may have the same and/or different shapes, configurations, and/or sizes. For example, a first jaw member and a second jaw member may include identical or substantially identical tissue contacting surfaces that are self-interfacing, but first jaw member may have a different body than second jaw member.

Securement of any of the components of the disclosed devices may be effectuated using known securement techniques such welding, crimping, gluing, heat-shrinking, fastening, etc.

Persons skilled in the art will understand that the structures and methods specifically described herein and shown in the accompanying figures are non-limiting exemplary embodiments, and that the description, disclosure, and figures should be construed merely as exemplary of particular embodiments. This disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of this disclosure, and that such modifications and variations are also included within the scope of this disclosure. Accordingly, the subject matter of this disclosure is not limited by what has been particularly shown and described.

What is claimed is:

1. A surgical instrument end effector, comprising:
a first jaw member having a first tissue contacting surface, the first tissue contacting surface configured to self-interface and including a first portion and a second portion, the first portion including a first plurality of teeth having a first tooth pitch, the second portion including a second plurality of teeth having the first tooth pitch, the second plurality of teeth offset from the first plurality of teeth by ½ of the first tooth pitch; and
a second jaw member including a second tissue contacting surface, the second tissue contacting surface positioned to enable the first and second tissue contacting surfaces to interface with one another.

2. The end effector of claim 1, wherein the first and second jaw members are movable relative to one another to cause the first and second tissue contacting surfaces to selectively interface with one another.

3. The end effector of claim 2, wherein the end effector defines a longitudinal axis and the first and second portions of the first tissue contacting surface are separated by a channel that extends longitudinally along the first jaw member.

4. The end effector of claim 1, wherein the plurality of teeth of the first portion of the first tissue contacting surface is spaced apart by a plurality of recesses defined in the first tissue contacting surface.

5. The end effector of claim 4, wherein the plurality of teeth of the second portion of the first tissue contacting surface are spaced apart by a plurality of recesses defined in the first tissue contacting surface.

6. The end effector of claim 3, wherein the first and second tissue contacting surfaces are substantially identical.

7. The end effector of claim 1, wherein the second tissue contacting surface includes first and second portions separated by a channel extending longitudinally along the second jaw member.

8. The end effector of claim 1, wherein the first jaw member defines a slot that extends through the first tissue contacting surface of the first jaw member.

9. The end effector of claim 1, wherein the first and second jaw members are identical or substantially identical.

10. The end effector of claim 2, wherein the first and second jaw members are configured to be manually controlled, robotically controlled, or combinations thereof.

11. An end effector for a robotically controlled surgical instrument, the end effector comprising:
a first jaw member having a first tissue contacting surface, the first tissue contacting surface being self-interfacing and including a first portion and a second portion, the first portion including a first plurality of teeth, the second portion including a second plurality of teeth, the first and second plurality of teeth have the same tooth pitch, the first plurality of teeth is longitudinally separated from the second plurality of teeth by ½ pitch offset; and
a second jaw member that includes a second tissue contacting surface that is identical or substantially identical to the first tissue contacting surface of the first jaw member, the second tissue contacting surface positioned to enable the first and second tissue contacting surfaces to interface with one another.

12. The end effector of claim 11, wherein the first and second jaw members are movable relative to one another to cause the first and second tissue contacting surfaces to selectively interface with one another.

13. The end effector of claim 12, wherein the end effector defines a longitudinal axis and the first and second portions of the first tissue contacting surface are separated by a channel that extends longitudinally along the first jaw member.

14. The end effector of claim 11, wherein the plurality of teeth of the first portion is spaced apart by a plurality of recesses defined in the first tissue contacting surface.

15. The end effector of claim 14, wherein the plurality of teeth of the second portion is spaced apart by a plurality of recesses defined in the first tissue contacting surface.

16. The end effector of claim 13, wherein the second tissue contacting surface includes a first portion and a second portion, the first portion of the first tissue contacting surface positioned to interface with the second portion of the second tissue contacting surface, the second portion of the first tissue contacting surface positioned to interface with the first portion of the second tissue contacting surface.

17. The end effector of claim 16, wherein the first and second portions of the second tissue contacting surface are separated by a channel that extends longitudinally along the second jaw member.

18. The end effector of claim 11, wherein the first jaw member defines a slot that extends through the first tissue contacting surface of the first jaw member.

19. The end effector of claim 11, wherein the first and second jaw members are identical or substantially identical.

20. A surgical instrument, comprising:
an end effector having a first jaw member and a second jaw member, each of the first and second jaw members includes a tissue contacting surface that is configured to self-interface and is separated by a longitudinal channel, the tissue contacting surfaces of the first and second jaw members positioned to interface with one another.
